(19) United States
(12) Patent Application Publication
(10) Pub. No.: US 2026/0060877 A1
(43) Pub. Date: Mar. 5, 2026

(54) FACE MASSAGING DEVICE

(71) Applicant: Amanda Ulumma Nwaopara, Miami Beach, FL (US)

(72) Inventor: Amanda Ulumma Nwaopara, Miami Beach, FL (US)

(21) Appl. No.: 19/317,900

(22) Filed: Sep. 3, 2025

Related U.S. Application Data
(60) Provisional application No. 63/689,912, filed on Sep. 3, 2024.

(51) Int. Cl. A61H 7/00; A61H 9/00; A61H 15/00; A61H 23/02; A61N 5/06

(52) U.S. Cl. CPC A61H 7/003; A61H 7/007; A61H 9/0078; A61H 15/0092; A61H 23/0254; A61N 5/0616; A61H 2015/0014; A61H 2201/0207; A61H 2201/10; A61H 2201/1246; A61H 2201/1604; A61H 2201/165; A61H 2201/1685; A61H 2201/5007; A61H 2201/5056; A61H 2205/022; A61H 2205/023; A61H 2205/025; A61H 2205/026; A61H 2205/028; A61H 2205/04; A61N 2005/0626; A61N 2005/0647; A61N 2005/0652; A61N 2005/0659; A61N 2005/0663

(57) ABSTRACT

A multifunctional, adaptive facial mask delivers cosmetic massage and light therapy in a hands-free format. The mask integrates three elements: (i) internal motion-guiding contours that channel tactile stimulation along anatomically relevant lymphatic paths, (ii) zone-selective actuation using vibration nodes, inflatable micro-bladders, or oscillating pads, and (iii) a flexible Light-Emitting Diode (LED) grid delivering red, near-infrared, and optional blue wavelengths. The soft, medical-grade silicone shell conforms to diverse facial anatomies and maintains a continuous smooth surface that isolates the user from hard mechanics. A microcontroller sequences stimulation across zones (forehead, temple, cheek, nasolabial, jawline, and neck strap) to emulate facial-yoga strokes and lymphatic drainage. Preset modes such as Lift, Recovery, and Glow manage timing, direction, and intensity. Optional enhancements include gentle heat, removable cooling inserts, and low-frequency micro-stimulation. The mask provides an integrated combination of guided internal geometry, sequenced hands-free actuation, and cosmetic light therapy within a flexible, inclusive design.

### FIELD OF THE INVENTION

The invention relates to a face massaging device with gua sha and roller movement or other movement to stimulate the skin. In particular, the invention relates to a hands-free lymphatic-massage facial mask with optional light and/or heat therapy.

### BACKGROUND

Light-Emitting Diode (LED) light therapy uses different light colors, like red and blue, to benefit the face by reducing wrinkles and improving skin texture through collagen stimulation (red light) and by clearing acne through killing bacteria and reducing oil production (blue light). The therapy also helps with wound healing, reducing redness, and improving the absorption of skincare products by penetrating the skin's layers.

Current Light-Emitting Diode (LED) masks are rigid, often uncomfortable, and lack massage or lymphatic stimulation. Manual devices such as rollers and gua sha require user effort and skill, are not programmable, and cannot deliver consistent directional motion. Post-procedure users and sensitive skin types need gentle, repeatable routines that blend light therapy with supportive tactile sequences.

Gua sha is a traditional Chinese medicine technique that involves scraping the skin with a smooth-edged tool to promote circulation and healing. The word “gua” means to scrape or scratch, while the word “sha” refers to reddish, elevated rashes, which sometimes appear after the technique is used. Gua sha can be used to help with a variety of health conditions, including musculoskeletal problems, tension headaches, migraines, neck pain, swelling, anxiety, fatigue, insomnia, and perimenopausal symptoms.

Gua sha, particularly in conjunction with a jade roller, can also be used for skincare to sculpt the jawline and cheekbones and reduce puffiness and soften wrinkles and fine lines. Specifically, gua sha as applied to the face in addition to improving overall health and radiance of the skin has a multitude of benefits such as contouring and sculpting facial features; promoting lymphatic drainage; reducing puffiness and fluid buildup; relieving facial and jaw tension; aiding circulation to give skin a glow; and firming and lifting the face over time.

To use gua sha, the user firmly strokes the skin in one direction with a gua sha tool. The scraping motion should be repeated until a light redness, called petechiae, appears, which indicates that blood is moving. Gua sha shouldn't be painful, but it can cause bruising.

For those that lack knowledge of the proper technique or the time to perform the movements as often, misuse of gua sha tools can be ineffective or potentially dangerous.

There therefore exists a need for an improved hands-free lymphatic-massage and LED facial mask.

### SUMMARY OF THE INVENTION

One aspect of the invention relates to a cosmetic wearable comprising a flexible substrate shaped to contact at least a portion of a user's face; surface features that define directional vector paths across the substrate; one or more actuation elements configured to generate shear and compression along the vector paths without handheld manipulation; optionally, one or more light-emitting devices configured to emit cosmetic light; and a controller configured to execute stored sequences to produce a perceptible wave of stimulation aligned to the vector paths and to coordinate light therapy provided by the light-emitting devices to supplement the stimulation.

Another aspect of the invention relates to a cosmetic method comprising contacting a user's face with a wearable having vector-defining surface geometry and, without requiring a handheld tool, generating directional shear and compression along upward and lateral paths followed by outward-then-downward drainage paths, optionally while emitting cosmetic light.

Yet another aspect of the invention relates to a kit comprising the wearable, at least one removable liner, at least one cooling or warming insert, and written instructions mapping routine names to anatomical vector paths.

In one embodiment, a face massaging device comprises a mask with at least one gua sha tool and at least one roller. Each of the at least one gua sha tool and the at least one roller automatically moves along a user's face.

The automated movement is controlled by a controller. Each of the at least one gua sha tool can move along a respective gua sha track on an inferior surface of the mask and each of the at least one roller can move along a respective roller track. Each of the at least one gua sha tool can be removably coupled to the respective gua sha track and each of the at least one roller can be removably coupled to the respective roller track.

In another embodiment, a face massaging device comprises: a mask with a flexible shell conforming to a plurality of facial geometries; at least one actuator disposed on the shell configured to deliver tactile stimulation; internal motion-guiding channels oriented to direct the tactile stimulation from the at least one actuator from a wearer's mid-face to pre-auricular and cervical regions; and an LED array positioned adjacent to at least one of the channels. A controller controls the at least one actuator to deliver the tactile stimulation that is then directed by the internal motion-guiding channels to generate wave-like stimulation. The controller also controls the LED array to provide light therapy that complements the wave-like stimulation.

The at least one actuator can comprise vibration motors located in the wearer's forehead, temple, cheek, nasolabial, jawline, and chin zones. The at least one actuator can also comprise inflatable micro-bladders fluidly coupled to a pump and valve array to generate sequential compression.

The controller can provide a plurality of preset massage and light therapy routines for the at least one actuator and the LED array. The LED array can emit red and near-infrared wavelengths through a flexible diffuser.

In an exemplary embodiment, the mask has an inner layer adhered or otherwise affixed to the shell. The inner layer can comprise a continuous silicone film that isolates the user from hard components. In this regard, the at least one actuator and the LED array can be positioned between the outer shell and the inner layer. The inner layer can have a skin-contacting surface, with the internal motion-guiding channels located on the skin-contacting surface.

To provide power, a rechargeable lithium-ion battery and a USB-C charging interface can be coupled to a charge-management integrated circuit. The USB-C interface accepts five-volt input from standardized sources and includes over-voltage, over-current, and thermal protection. The USB-C interface can have a receptacle that is recessed and sealed by an elastomeric gasket that maintains splash resistance. Firmware can limit heat and high-intensity modes while external power is connected.

A method of cosmetic treatment comprises placing the face massaging device on a user, initiating a program that sequences stimulation from cheek to pre-auricular to cervical regions, and concurrently emitting light from the LED array.

### BRIEF DESCRIPTION OF THE DRAWING

A more complete understanding of the present invention, and the attendant advantages and features thereof, will be more readily understood by reference to the following description when considered in conjunction with the accompanying drawing wherein:

FIG. 1 schematically shows an embodiment of a face massaging device according to the invention.

FIG. 2 shows a front view of another embodiment of a face massaging device on user, showing zone outlines.

FIG. 3 shows a side view of the device of FIG. 2.

FIG. 4 is an interior plan view of the device of FIG. 2 showing the motion-guiding channels.

FIG. 5 is an exploded assembly view of layers, including charge board, battery, and gasketed USB-C receptacle.

FIG. 6 schematic shows the device of FIG. 2 with vibration-based actuators.

FIG. 7 schematic shows the device of FIG. 2 with a pneumatic bladder layout with sequencing arrows.

FIG. 8 shows an oscillating pad mechanism.

FIG. 9 is a firmware flow diagram for a mode of operation.

### DETAILED DESCRIPTION

As required, embodiments are disclosed herein; however, it is to be understood that the disclosed embodiments are merely examples and that the devices and methods described below can be embodied in various forms. Therefore, specific structural and functional details disclosed herein are not to be interpreted as limiting, but merely as a representative basis for teaching one skilled in the art to variously employ the present subject matter in virtually any appropriately detailed structure and function. Further, the terms and phrases used herein are not intended to be limiting, but rather, to provide an understandable description of the concepts.

It can be advantageous to set forth definitions of certain words and phrases used throughout this disclosure. The terms “a” or “an”, as used herein, are employed to describe elements and components described herein. This is done merely for convenience and to give a general sense of the scope of the disclosure. This description should be read to include one or at least one and the singular also includes the plural unless it is obvious that it is meant otherwise. The term plurality, as used herein, is defined as two or more than two. The term another, as used herein, is defined as at least a second or more. The term “coupled,” as used herein, is defined as “connected,” although not necessarily directly, and not necessarily mechanically.

The term “communicate,” as well as derivatives thereof, encompasses both direct and indirect communication. The terms “include” and “comprise,” as well as derivatives thereof, mean inclusion without limitation. The term “or” is inclusive, meaning and/or. The phrase “associated with,” as well as derivatives thereof, can mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items can be used, and only one item in the list can be needed. For example, “at least one of: A, B, and C” includes any of the following combinations: A; B; C; A and B; A and C; B and C; and A, B, and C.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms “substantial” and “substantially” means, when comparing various parts to one another, that the parts being compared are equal to or are so close enough in dimension that one skill in the art would consider the same. Substantial and substantially, as used herein, are not limited to a single dimension and specifically include a range of values for those parts being compared. The range of values, both above and below (e.g., “+/−” or greater/lesser or larger/smaller), includes a variance that one skilled in the art would know to be a reasonable tolerance for the parts mentioned.

Note that not all of the activities described in the general description or the examples are required, that a portion of a specific activity may not be required, and that one or more further activities can be performed in addition to those described. Still further, the order in which activities are listed are not necessarily the order in which they are performed.

The invention generally relates to a face massaging device designed to revolutionize skincare routines by integrating automatic gua sha and roller movements or other stimulating movements into a hands-free device. Unlike traditional handheld tools currently available in the market, this innovative face mask offers a convenient and efficient way to enhance skincare without the need for manual manipulation.

Equipped with built-in mechanisms that generate gentle, oscillating motions, the face mask mimics the techniques of gua sha and facial rolling. These movements are specifically tailored to promote lymphatic drainage and increase blood flow to the skin, resulting in improved circulation and a rejuvenated complexion.

One of the notable features of this design is its hands-free functionality. Users simply wear the face mask on oiled skin, allowing the automated movements to be effective without the need for additional manual effort. This not only enhances user convenience but also ensures consistent and effective skincare treatment. Another distinctive feature of this design is its adaptability through customizable settings for different target areas. These settings enable precise targeting of distinct anatomical regions, including the nasolabial folds, frontal region, glabellar lines, and sculpted jawline.

The inspiration for this innovative concept stems from the transition from manual to electric toothbrushes, where automated technology significantly improved oral hygiene routines. Similarly, this automated face mask aims to set a new standard in skincare technology, offering users a more convenient and efficient way to achieve healthy, glowing skin at home.

In summary, the novel face mask with automatic gua sha and rollers movement represents a groundbreaking advancement in skincare technology. By combining innovative design with automated functionality, it aims to provide users with a hassle-free solution for enhancing their skincare routine and achieving optimal skin health.

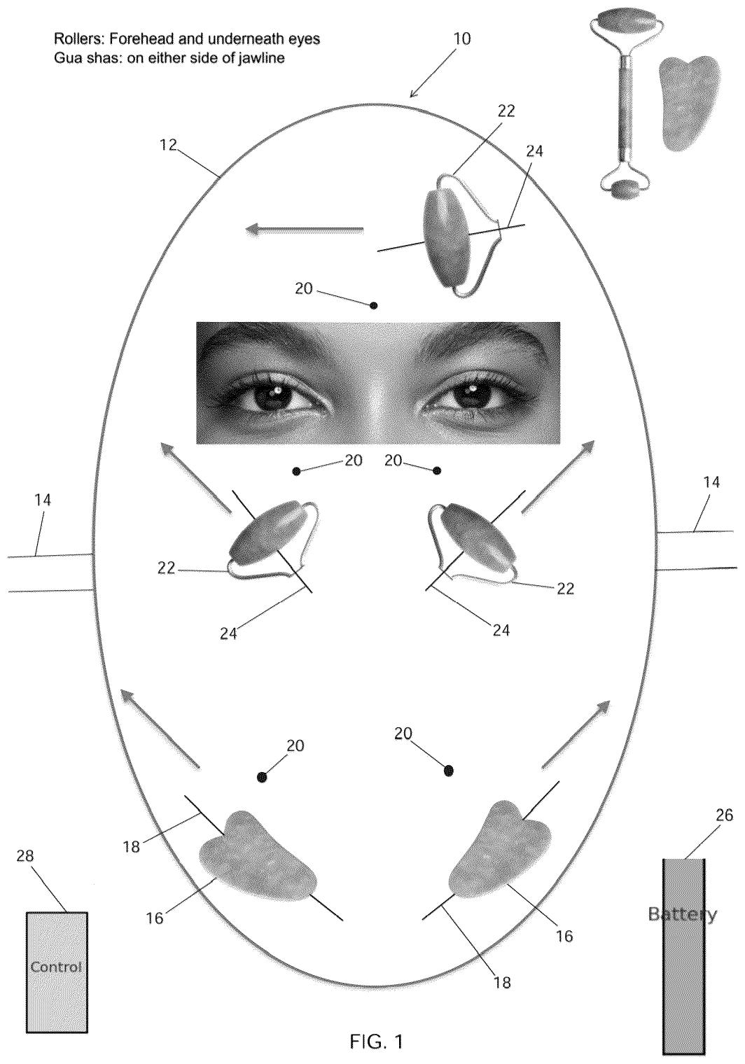

With reference to FIG. 1, a first embodiment of a face massaging device 10 includes a mask 12 that substantially covers the entire face. Mask 12 is similar to a traditional sheet mask but has some rigidity to hold the other components of face massaging device 10. Mask 12 is made of a soft, flexible material like silicone, gel, or plastic, which are designed to conform comfortably to the face's contours. In this regard, mask 12 can come in a molded shape, but can be further moldable to provide a better fit to a user's face. Mask 12 can be provided with straps 14 to removably secure mask 12 to a user's face. The present invention contemplates other means to removably secure mask 12 to a user's face.

The side of mask 12 that abuts the face of a user when device 10 is in use includes one or more gua sha tools 16 that move along a track 18. This movement is achieved through a combination of motorized tracks and battery-powered operation. Small motors embedded within the mask drive gears connected to the tracks 18, allowing the gua sha tools 16 to simulate manual scraping and rolling actions. The speed, intensity, and direction of the tools are controlled via a control interface 28, which could be a handheld remote or smartphone app. The mask is powered by a rechargeable battery pack 26 that supplies energy to the motors, ensuring smooth and consistent motion along the face's contours. The tracks and guides keep the tools aligned, while adjustable pressure settings ensure a comfortable and effective massage experience.

Mask 12 can include an array of LEDs 30 to provide for light therapy, if desired. If present, LEDs 30 can be powered by battery pack 26 and controlled with control interface 28.

Although FIG. 1 shows two gua sha tools 16 on either side of the jawline, the present invention contemplates different numbers and/or different locations of gua sha tools 16 and their respective tracks 18. In an exemplary embodiment, each gua sha tool 16 moves back and forth in both directions along their respective track 18. When moving in a first direction (e.g. upward), more pressure (compared to the second direction) is applied by gua sha tool 16 and the movement is slower (compared to the second direction) to maintain contact with the user's skin. When moving in a second direction (e.g. downward), the movement of gua sha tool 16 is faster with less pressure so that gua sha tool 16 barely touches the skin. This cycle of movement can be repeated. In this regard, one or more sensors 20 for detecting light and/or heat can be positioned proximal to track 18 to assist in determining if the cycle of movement needs to be adjusted or stopped.

The side of mask 12 that contacts the user's face incorporates rollers 22 that move along tracks 24, as well as gua sha tools 16. The movement of these tools is achieved through small motors embedded within the mask's structure, which drive gears connected to the tracks 18. This setup allows the rollers and gua sha tools to simulate manual scraping and rolling actions. The device is powered by a battery pack 26, either integrated or externally connected, providing the necessary energy for continuous operation. This battery pack is rechargeable, offering convenience and cost-effectiveness for multiple uses.

Users have the ability to control the intensity, speed, and pattern of the tool movements via a control unit 28, which could be a handheld remote or smartphone app. This control system allows for customizable settings, enabling personalized facial massage experiences. Additionally, the mask features programmable settings, offering preset routines or customizable programs to guide the movement of the rollers and gua sha tools across the face. To ensure smooth and consistent motion, the tracks 18 are designed to guide the tools along the contours of the face, with integrated guides to maintain alignment and prevent deviation. The pressure applied by the tools is adjustable through either mechanical springs or electronically controlled actuators, ensuring a comfortable and effective massage tailored to the user's preferences. Although FIG. 1 shows three rollers 22 (one on the forehead and two underneath the eyes), the present invention contemplates different numbers and/or different locations of rollers 22 and their respective tracks 24.

In an exemplary embodiment, each roller 22 moves back and forth in both directions along their respective track 24. As was the case with gua sha tool 16, the speed and/or pressure can be the same in both directions or can be different. This cycle of movement can be repeated. In this regard, one or more sensors 20 for detecting light and/or heat can be positioned proximal to track 24 to assist in determining if the cycle of movement needs to be adjusted or stopped.

The present invention contemplates that gua sha tools 16 are removably coupled to their respective tracks 18 and rollers 22 are removably coupled to their respective tracks 24. The present invention also contemplates that gua sha tools 16 can be removably coupled to tracks 24 and rollers 22 can be removably coupled to tracks 18. This further increases the versatility of device 10 to better suit a user's needs.

Each of gua sha tools 16 and rollers 22 can include a motor for enabling the movement and can be powered individually by a battery. Alternatively, gua sha tools 16 and rollers 22 can be powered by one battery 26, which is preferably a rechargeable battery.

Device 10 includes a controller 28 for controlling the movements of gua sha tools 16 and rollers 22. Controller 28 can be a device that is wired to mask 12. In some embodiments, controller 28 is positioned on mask 12. In other embodiments, controller 28 is remote from mask 12. Additionally, controller 28 can interface to a mobile phone or computer app using a Bluetooth, Wi-Fi, or similar mechanism.

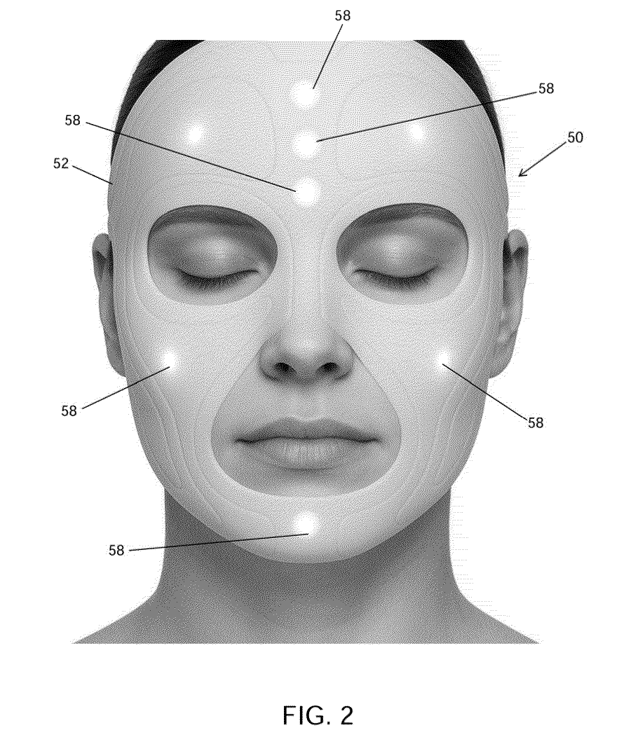

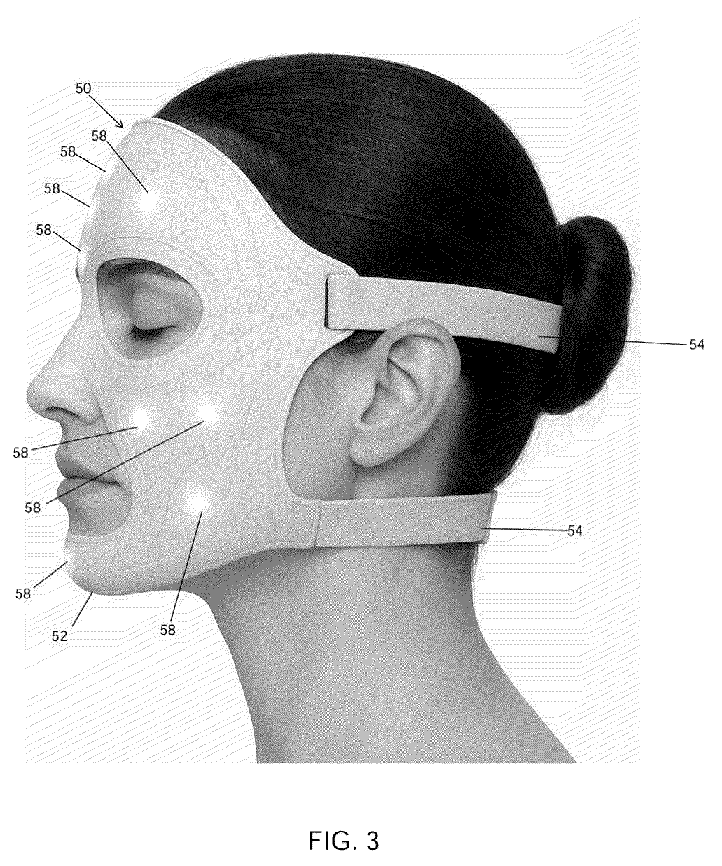

With reference to FIGS. 2-9, another embodiment of a face massaging device 50 is shown. Device 50 is a multifunctional, adaptive facial mask that delivers cosmetic massage and light therapy in a hands-free format. The mask integrates three elements: (i) internal motion-guiding contours that channel tactile stimulation along anatomically relevant lymphatic paths, (ii) zone-selective actuation using vibration nodes, inflatable micro-bladders, and/or oscillating pads, and (iii) a flexible Light-Emitting Diode (LED) grid delivering at least one of red, near-infrared, and blue wavelengths. The soft, medical-grade silicone shell conforms to diverse facial anatomies and maintains a continuous smooth surface that isolates the user from hard mechanics. A microcontroller sequences stimulation across zones (forehead, temple, cheek, nasolabial, jawline, and neck strap) to emulate facial-yoga strokes and lymphatic drainage. Preset modes such as Lift, Recovery, and Glow (detailed below) manage timing, direction, and intensity. Optional enhancements include gentle heat, removable cooling inserts, and low-frequency micro-stimulation.

Mask 52 is similar to a traditional sheet mask but has some rigidity to hold the other components of face massaging device 50. Mask 52 is made of a soft, flexible material like silicone, gel, or plastic, which are designed to conform comfortably to the face's contours. In this regard, mask 52 can come in a molded shape, but can be further moldable to provide a better fit to a user's face.

Device 50 includes a mask 52 that substantially covers the entire face. Mask 52 can be provided in different sizes (e.g. adult, children, etc.), but in general, mask 52 can be provided in one-size-fits-most design and used with adaptive cheek pads, chin pads, etc. to account for anatomical anomalies and/or customize fit to a user's preference.

Additionally, mask 52 can be provided with straps 54 to removably secure mask 52 to a user's face. The present invention contemplates other means (e.g. adhesives, magnets, etc.) to removably secure mask 52 to a user's face.

The side of mask 52 that abuts the face of a user when device 50 is in use includes a plurality of channels 56 that direct tactile vectors along facial paths. Channels 56 are formed by ridges 57. Channels 56 and ridges 57 are configured dimensioned and arranged so that in conjunction with actuators (described below), directional, hands-free waves aligned to lymphatic drainage and facial-yoga strokes are generated.

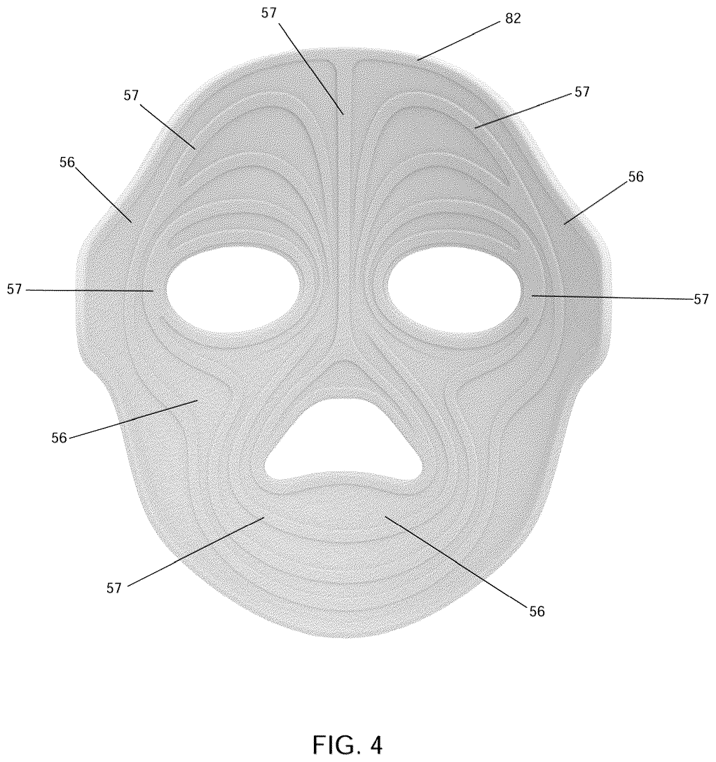

The pattern of channels 56 and the pattern of ridges 57 are best seen in FIG. 4. The pattern is based on known lymphatic drainage and facial-massage stroke directions. These anatomical vectors were translated into smooth internal grooves, with dimensioned (width and depth) selected so the movement of the actuators produces a coherent upward-then-downward wave rather than random vibration.

The side of mask 52 that abuts the face of a user when device 50 is in use also includes one or more arrays of LEDs 58 that deliver light therapy via at least one of red, near-infrared, and blue wavelengths. If desired, a diffuser to be added to soften or otherwise adjust the emitted wavelengths. It should be noted that the location and number of LED arrays 58 shown in the figures is only representative and should be considered non-limiting. In an exemplary embodiment, LED arrays 58 are co-extensive with the actuation zones, i.e. the areas on the user's face to be treated.

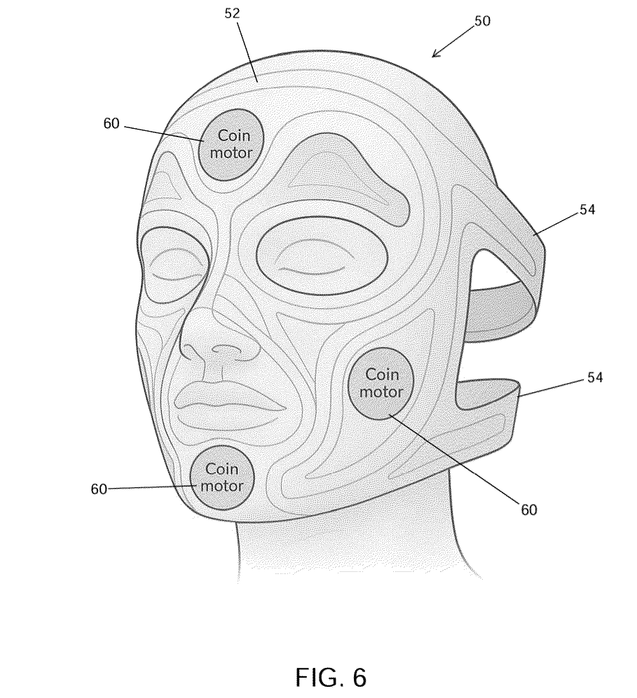

FIG. 6 shows one embodiment of the actuators to generate tactile stimulation that is directed by channels 56. In particular, coin motors 60 vibrate when activated, and the vibratory energy generated is steered by the geometry of channels 56 to create a wave effect. Although FIG. 6 shows coin motors 60 in cheek, nasolabial, temple, and jawline regions, additional and/or different locations and/or number of coin motors 60 are contemplated by the invention.

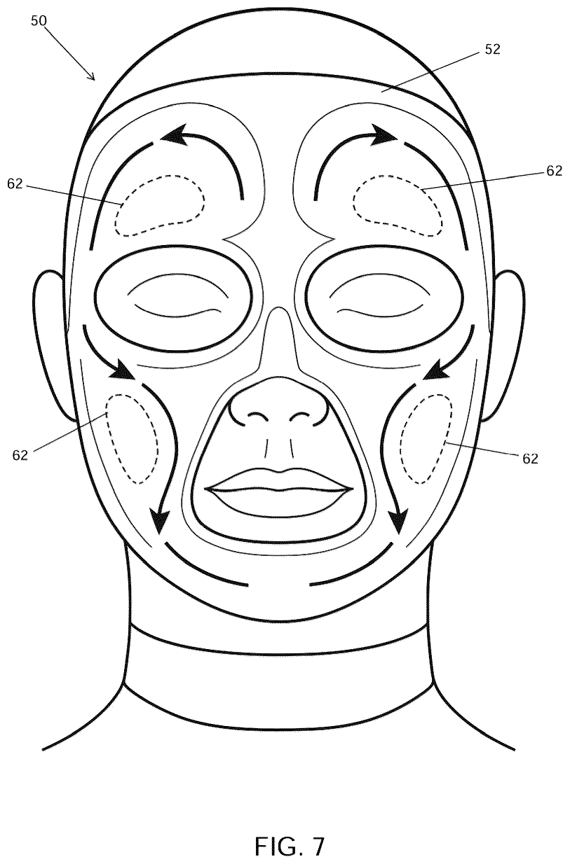

FIG. 7 shows another embodiment of the actuators to generate tactile stimulation that is directed by channels 56. Specifically, sequentially inflated micro-bladders 62 generate outward-then-downward compression as illustrated by the arrows. Inflation and deflation of micro-bladders 62 is operated by a micro-pump with a valve array. Although FIG. 7 shows micro-bladders 62 above and below each eye, additional and/or different locations and/or number of micro-bladders 62 are contemplated by the invention.

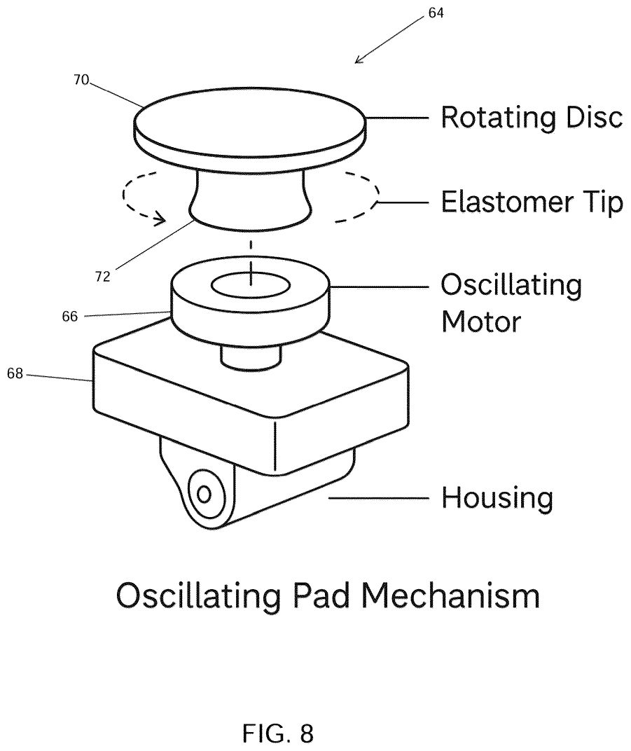

FIG. 8 shows yet another embodiment of the actuators to generate tactile stimulation that is directed by channels 56. Specifically, actuators are in the form of one or more oscillating pads 64. Each oscillating pad 64 includes a motor 66 in a housing 68 that rotates a disc 70 on an elastomer tip 72. Disc 70 on tip 72 delivers kneading and lifting stimulation, which is directed by channels 56. The locations and numbers of oscillating pads 64 can match those of coin motors 60 of FIG. 6. However, additional and/or different locations and/or number of oscillating pads 64 are contemplated by the invention.

The invention also contemplates actuators that include any combination of coin motors 60, micro bladders 62, and oscillating pads 64. In other words, there is a combination of vibration, pneumatic, and oscillating elements.

Regardless of whether the actuators are coin motors 60, micro bladders 62, oscillating pads 64, or some combination thereof, the speed, intensity, and direction of the actuators are controlled via a control unit 74, which could be a handheld remote or smartphone app. The mask is powered by a rechargeable battery pack 76 that supplies energy to the actuators, ensuring smooth and consistent motion along the face's contours. Battery pack 76 can be either integrated or externally connected, providing the necessary energy for continuous operation. This battery pack can be rechargeable via charging port 78, offering convenience and cost-effectiveness for multiple uses. Charging port 78 can be sealed by an elastomeric gasket that maintains splash resistance.

Users have the ability to control the intensity, speed, and pattern of the actuator activation via control unit 74, which could be a handheld remote or smartphone app. This control system allows for customizable settings, enabling personalized facial massage experiences. Additionally, the mask features programmable settings, offering preset routines or customizable programs.

Control unit 74 can be a device that is wired to mask 52. In some embodiments, control unit 74 is positioned on mask 52. In other embodiments, control unit 74 is remote from mask 52. Additionally, control unit 74 can interface to a mobile phone or computer app using a Bluetooth, Wi-Fi, or similar mechanism.

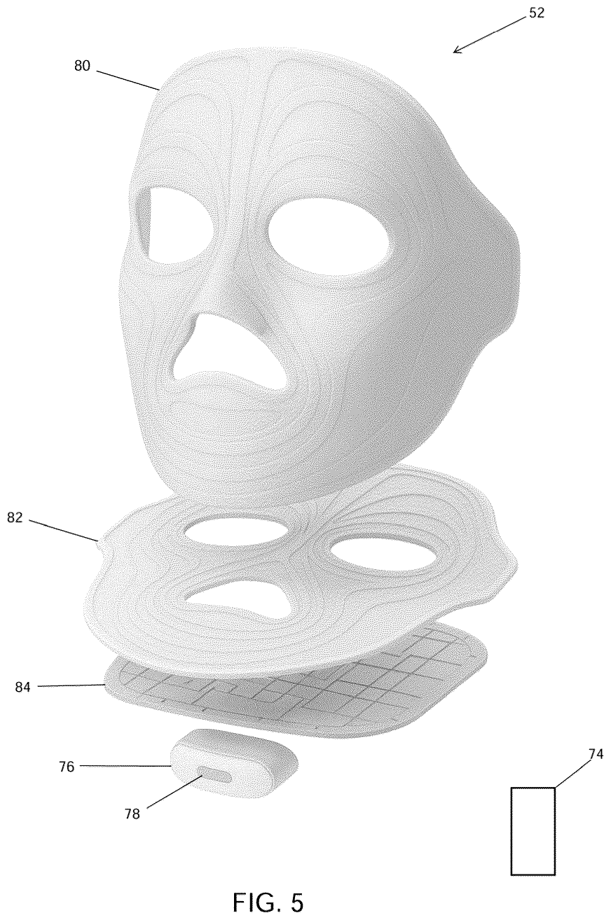

FIG. 5 shows an exemplary embodiment of mask 52. Mask 52 comprises an outer layer 80 and an inner layer 82. Inner layer 82 forms a continuous skin-contact layer that conceals and insulates the hardware, i.e. the actuators (coin motors 60, micro bladders 62, oscillating pads 64, or some combination thereof) and the associated electronic circuitry 84. Inner layer 82 can be hypoallergenic, latex-free silicone for skin contact so that there is smooth silicone against skin with no hard parts felt. Any suitable material could be used instead of silicone. Non-limiting examples include thermoplastic elastomer, polyurethane, hydrogel, thermoplastic polyurethane film, or combinations thereof.

As inner layer 82 contacts skin, inner layer 82 would be provided with channels 56 (which are formed by ridges 57) on one side. The other side of inner layer 82 would be bonded or otherwise attached to outer layer 80 with the hardware therebetween.

### Preset Modes

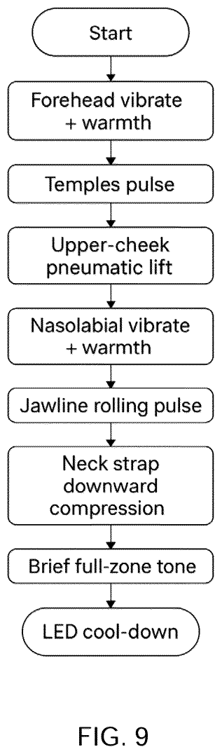

The controller (control unit 74) for either device 10 or device 50 can be programmed by the user or the user can select preset modes. Examples of these modes include the following.
Lift Mode (approximately 8-12 minutes): Slow pneumatic lift on cheeks with gentle knead at nasolabial sites, rolling jawline wave, brief full-face tone, light-emitting diode cool-down. Forehead warmth and vibration, temples pulse, upper-cheek pneumatic lift, nasolabial vibration, jawline rolling pulse, neck-strap downward compression, brief full-zone tone, light-emitting diode cool-down. FIG. 9 shows the firmware flow diagram for this Lift Mode.
Recovery Mode: No knead, cooler surface, 10-18 millimeter mercury (mmHg) waves only, low light-emitting diode settings. Reduced intensity with longer holds, outward-then-downward sequences, low-power light-emitting diodes, cooler surface.
Glow Mode: Short session with light vibration and light-emitting diodes, minimal compression. Shorter routine with higher light-emitting diode duty cycle and light vibration.

### Sensations Experienced by User

Representative examples of what use of either device 10 or device 50 feels like to the wearer include.

Cheeks and jaw—A slow wave of pressure that travels outward and upward, then a gentle release.

Nasolabial folds and brow—Small kneading or rolling pulses that soften tension.

Temples and neck—Calming drainage pulses that move fluid downward through the strap to the neck.

Overall—Smooth silicone against skin with no hard parts felt. Optional mild warmth creates a spa-like feel while light-emitting diodes add a soft glow.

Achieving these feelings will depend on the user as well as the specific configuration of device 10 or device 50 (e.g. coin motor vs. bladders vs. oscillating pads). Suggested starting specifications, from which the user can adjust to achieved the desired effect include:
Pneumatic wave: Inflate, hold, and deflate over three to six seconds per zone with 15-30 millimeter mercury (mmHg) peak pressure; sequence cheek→temple→jaw→neck.
Kneading pads: 1-3 millimeter stroke at 0.5-1.5 hertz for a slow, hand-like feel.
Vibration layer: 80-120 hertz at light amplitude to add tactile texture and avoid buzzy sensations above 200 hertz.
Heat cap: Surface temperature at or below 40 degrees Celsius; Recovery Mode favors cooling only.
Automatic shutoff: 12-15 minutes; target noise below 40 decibels at ten centimeters distance.
Charging behavior: While on external USB-C power, limit to cooler profiles unless explicitly overridden.

### Safety and Compliance

Exemplary embodiments of either device 10 or device 50 can include safety features such as:
Time and thermal cutoffs with maximum surface temperature less than or equal to 40 degrees Celsius.
Zone-specific pressure limits and watchdog systems for pressure and temperature.
Hypoallergenic, latex-free silicone for skin contact.
Removable, washable liner; ingress-protection-rated compartment for electronics.
Power safety: battery protection circuit with dual integrated circuit cutoff and positive temperature coefficient resettable fuse; negative temperature coefficient thermistor-based charge monitoring; conformal coating around the charge board.
Position all text and marketing as cosmetic use only.

### Exemplary Dimensions and Ranges

Exemplary embodiments of either device 10 or device 50 can include a mask that uses medical-grade platinum-cure silicone with a global shell thickness between 1.8 and 2.4 millimeters, while comfort zones over the zygoma and mandible increase to 2.6 to 3.2 millimeters to distribute load evenly.

The base shell targets a Shore A durometer of 20 to 30 for structure, and the continuous contact liner is a softer silicone film at 0.6 to 1.0 millimeter thickness with an effective feel of Shore 00 forty to sixty for skin comfort. Channels that guide motion are cut into an intermediate layer to a depth of 0.6 to 1.2 millimeters and a width of 3 to 8 millimeters with at least 1.0 millimeter fillets to avoid pressure peaks. Eye apertures span 35 to 44 millimeters on the major axis and 22 to 28 millimeters on the minor axis with a 1.5 to 2.0 millimeter edge radius, and a nasal bridge relief of 1.5 to 2.5 millimeters maintains fit and airflow.

The primary straps are elastic knit bands 18 to 25 millimeters wide with a micro-adjust buckle and a circumference range of 120 to 230 millimeters. Pneumatic elements use thermoplastic polyurethane bladder film from 0.10 to 0.18 millimeters with heat-seal beads 1.5 to 2.0 millimeters wide and a burst pressure of at least 120 millimeters mercury. Vibration actuators employ coin motors with a 10 to 12 millimeter footprint and an isolation gasket of 0.8 to 1.2 millimeters so the user never contacts hard parts. Optional oscillating pads present a low profile with a disc diameter of 12 to 16 millimeters and a total stack height no more than 5.5 millimeters above the substrate, while the elastomer tip is tuned in the Shore 00 thirty to forty-five range for a hand-like feel.

In a concrete working example, the shell is 2.0 millimeters, the channels are 0.8 millimeters deep and 4 to 6 millimeters wide, the coin motors are 10 millimeter units with 1.0 millimeter silicone isolation, and the cheek and temple bladders are fabricated from 0.12 millimeter thermoplastic polyurethane.

### Exemplary Electronics Specification

In exemplary embodiments of either device 10 or device 50, power is supplied by a single-cell lithium-ion pouch battery of 1,800 to 2,200 milliampere-hours, with a best-mode capacity of 2,000 milliampere-hours yielding approximately 60 to 75 minutes in Lift Mode, 90 to 110 minutes in Recovery Mode, and 110 to 140 minutes in Glow Mode under typical loads. Charging occurs through a recessed USB-C receptacle at five volts with a 1.2 to 1.6 ampere charge current and charge control guided by the Japan Electronics and Information Technology Industries Association negative temperature coefficient thermistor standard. The practical charge time from empty to full is about 1.8 to 2.3 hours at 1.5 amperes.

The LED array delivers cosmetic wavelengths at 630±10 nanometers for red and 850±20 nanometers for near-infrared, with an optional blue at 465±15 nanometers. Irradiance at the skin after diffusion is adjustable from 10 to 25 milliwatts per square centimeter for red, 15 to 30 milliwatts per square centimeter for near-infrared, and 5 to 12 milliwatts per square centimeter for blue. Duty cycles run at 30 to 60 percent in Lift Mode, 15 to 30 percent in Recovery Mode, and 60 to 80 percent in Glow Mode.

Vibration overlays operate at 80 to 120 hertz with 0.2 to 0.6 g effective amplitude using bursts of 250 to 600 milliseconds separated by 150 to 300 milliseconds rests. Pneumatic compression peaks at 15 to 30 millimeters mercury with three- to six-second inflate-hold-deflate cycles per zone and 200 to 500 millisecond inter-zone delays to create a perceived wave. Thermal controls cap the outer surface at or below 40 degrees Celsius with throttling above 38 degrees Celsius under negative temperature coefficient thermistor feedback, and acoustic output remains under 40 decibels at ten centimeters in Lift Mode.

The power path includes over-voltage, over-current, and thermal protections. The USB-C port is sealed with an elastomer gasket to achieve splash resistance consistent with Ingress Protection Code 4 (protection against splashing water).

### Exemplary Manufacturing Details

Early production runs follow a laminate stack strategy that bonds a structural silicone layer, a channel layer, an actuation layer, a detachable flexible light-emitting diode sheet, and the continuous liner using medical-grade pressure-sensitive adhesive or room-temperature vulcanizing silicone for silicone-to-silicone interfaces.

If the actuators include inflatable bladders, the pneumatic set is formed by heat-sealing thermoplastic polyurethane films on steel tooling, while the low-noise micro-pump and latching valve array mount to a compact carrier with strain-relieved tubing.

Light-emitting diode drive uses constant-current drivers on a two-layer flexible polyimide printed circuit board with a diffuser film. The controller resides on a small four-layer rigid or rigid-flex printed circuit board that also hosts the charge-management integrated circuit, battery protection system, and battery fuel gauge.

The USB-C receptacle sits in a recessed pocket with a molded silicone gasket, a downward drainage path, and conformal coating around the charge board. Once geometry is finalized, the design can migrate to selective overmold for improved repeatability while keeping the light-emitting diode sheet and liner serviceable.

The target ingress performance is splash resistant so cleaning can be done without soaking, and the liner and light-emitting diode sheet are designed to be removable for washing or replacement.

### Exemplary Firmware Parameters

Either device 10 or device 50 runs a simple state machine with the following states: Idle, Mode Select, Active, Charging, and Fault. Features are gated according to power and temperature conditions.

Zones are mapped as follows: Zone 1 (forehead), Zone 2 (temples), Zone 3 (upper cheek), Zone 4 (nasolabial), Zone 5 (jawline), and Zone 6 (neck strap).

In a directly implementable Lift routine (see FIG. 9), the program warms the forehead at 37 degrees Celsius with 100 hertz vibration for thirty seconds, pulses the temples at 90 hertz for thirty seconds, applies a 22 millimeter mercury (mmHg) pneumatic lift to the upper cheek for 4.5 seconds, overlays 100 hertz bursts at the nasolabial zone for three cycles, runs a rolling jawline pulse at 18 millimeters mercury for 3.5 seconds with a synchronized 100 hertz sweep, and finishes with a neck strap downward compression at 12 millimeters mercury for three seconds before a one-second full-zone tone and a low-duty light-emitting diode cool-down.

Safety interlocks disable heat if the negative temperature coefficient thermistor exceeds 38 degrees Celsius, pause all actuation if pressure readings drift beyond ±3 millimeters mercury of the target, and restrict the device to Recovery and Glow Modes while external power is detected. A long-press plus application confirmation can re-enable Lift Mode during charging if the user opts in, although the default behavior remains conservative.

Representative Variations As previously mentioned, the embodiments of device 10 and device 50 are illustrative.

The following non-limiting variations and equivalents are expressly contemplated and form part of this disclosure.
1) Surfaces, Geometry, and Vectoring
Guided surfaces: The wearable substrate may include channels, ribs, ridges, nodules, combs, domes, fins, or textured fields that define vector paths for shear, lift, and compression.
Edge-like features: Smooth or micro-rounded edges configured to produce directional shear functionally equivalent to gua sha strokes without requiring a handheld tool.
Gradient stiffness: Zones of differing hardness or modulus to steer force laterally and upward, or outward-then-downward, including multi-layer laminates and inserts.
Coverage: Face, scalp, periauricular region, neck, and upper chest (décolleté); full-face masks, half-masks, jawline wraps, temple bands, neck straps, or modular panels.
Fit systems: Straps, magnets, adhesive interfaces, suction features, or anatomical undercuts; single-size or multi-size systems; removable pads, shims, or wedges.
2) Materials
Skin-contact layer formed from silicone, thermoplastic elastomer, polyurethane, hydrogel, thermoplastic polyurethane film, or combinations thereof.
Structural layers formed from silicone, ethylene-vinyl acetate, polyurethane, polyamide, polyester, or fiber-reinforced elastomers.
Textures and inserts formed from elastomer, gel, microcellular foam, or phase-change composites.
Disposable or semi-disposable liners, shields, and covers.
3) Actuation Families
Mechanical: Rotary or linear vibration, linear resonant, cam-driven or eccentric mass mechanisms, oscillating pads, rolling elements.
Fluidic: Pneumatic or hydraulic micro-bladders; peristaltic or manifolded networks; elastomer bellows; liquid or air displacement.
Electroactive and smart materials: Piezoelectric stacks or films, dielectric elastomer actuators, shape-memory alloys or polymers, magnetorheological or electrorheological elements.
Thermal expansion: Wax, gel, or fluid expansion to create stroke.
Magnetic drive: Permanent magnet and coil interactions to pulse or sweep contact tips.
Hybridization: Any combination of the above within one device.
4) Sequencing and Motion Patterns
Wave forms: Sequential, overlapping, counter-propagating, randomized, or user-selected paths; continuous or pulsed.
Kinematics: Shear, knead, tap, roll, lift, glide, and press patterns; net vector fields aligned to lymphatic drainage or muscular lines of pull.
Adaptivity: Intensity, frequency, duty cycle, and path altered by user input, sensed data, or preset routines.
Manual-assist variant: Wearable geometry that converts head or jaw movement into guided shear along the same vector paths (non-powered embodiment).
5) Light and Other Energy Modalities
Light therapy: Light-emitting devices producing wavelengths from 400 to 1,000 nanometers; continuous or pulsed; fixed, detachable, or replaceable panels; diffused or direct emission.
Alternative emitters: Organic light-emitting devices, micro-light-emitting devices, laser diodes with diffusers, or phosphor-converted emitters.
Additional cosmetic energies: Non-therapeutic microcurrent or electrical stimulation, low-intensity ultrasound, thermal warming or cooling, vibroacoustic cues.
Layering: Any energy modality used alone or in combination with mechanical actuation.
6) Sensing and Feedback (Cosmetic, Non-diagnostic)
Sensed variables: Temperature, pressure, flow or vacuum level, motion or orientation, contact quality, optical reflectance, or skin hydration proxies.
Use: To gate safety, personalize sequences, log usage, or adapt intensity in real time.
7) Control and Interfaces
User inputs: Mechanical switches, capacitive touch, squeeze sensors, or voice prompts; local control or companion application control.
Connectivity: Short-range wireless connection for control, logging, and software updates; or no wireless in a fully self-contained device.
Automation: Preset libraries that simulate manual techniques including upward strokes, outward glides, multi-point kneads, and drainage sequences.
8) Power, Charging, and Safety
Cells: Lithium-ion, lithium-polymer, nickel-metal hydride, primary cells in disposable variants, or external power.
Charging: Universal Serial Bus Type-C, inductive wireless charging, magnetic pin connectors, or docking cradles.
Protections: Over-voltage, over-current, thermal, short-circuit, and usage lockouts tied to temperature or contact sensors.
Ingress and serviceability: Splash-resistant compartments; removable or washable liners; user-replaceable light panels or pads.
9) Cosmetic Methods of Use (to capture “facial yoga” and “gua sha” spaces)
A cosmetic method comprises: placing a wearable substrate on the face; executing directional sequences that produce net upward and lateral shear and compression along predefined paths; optionally emitting non-therapeutic light; and ending with outward-then-downward pulses that emulate lymphatic drainage.
The sequences may be defined by a stroke library including lift, glide, knead, roll, and press patterns that functionally replicate manual gua sha or facial-yoga techniques without requiring a handheld tool.
10) Kits and Articles of Manufacture
Kits comprising the wearable device, removable liners, cooling or warming inserts, contact gel, and a quick-start guide that maps routine names to vector paths.
Replacement light panels, disposable pads, or removable actuator modules.
11) Manufacturing Variants
Processes including lamination, overmolding, insert molding, heat sealing, ultrasonic welding, laser micromachining, water-jet cutting, die cutting, three-dimensional printing (stereolithography or digital light processing) for molds or direct parts, and chemical or plasma surface treatments for adhesion.
Geometry formed by molding, machining, three-dimensional printing, or a combination.
12) Non-Face Wearables Covering Design-Arounds
Equivalent wearables for scalp, neck, shoulders, chest, or multi-region wraps using the same vectorized surface geometry and sequenced actuation with optional light.
Narrow strips or pads positioned along jawline, nasolabial folds, temples, or neck that still implement the claimed vector fields.

All references cited herein are expressly incorporated by reference in their entirety. It will be appreciated by persons skilled in the art that the invention is not limited to what has been particularly shown and described herein above. In addition, unless mention was made above to the contrary, it should be noted that all of the accompanying drawings are not to scale. There are many different features to the invention and it is contemplated that these features may be used together or separately. Thus, the invention should not be limited to any particular combination of features or to a particular application of the invention. Further, it should be understood that variations and modifications within the spirit and scope of the invention might occur to those skilled in the art to which the invention pertains. Accordingly, all expedient modifications readily attainable by one versed in the art from the invention set forth herein that are within the scope and spirit of the present invention are to be included as further embodiments of the invention.

The description in the present application should not be read as implying that any particular element, step, or function is an essential or critical element that must be included in the claim scope. The scope of patented subject matter is defined only by the allowed claims. Moreover, none of the claims invokes 35 U.S.C. § 112(f) with respect to any of the appended claims or claim elements unless the exact words “means for” or “step for” are explicitly used in the particular claim, followed by a participle phrase identifying a function.

Benefits, other advantages, and solutions to problems have been described above with regard to specific embodiments. However, the benefits, advantages, solutions to problems, and any feature(s) that can cause any benefit, advantage, or solution to occur or become more pronounced are not to be construed as a critical, required, sacrosanct or an essential feature of any or all the claims.

After reading the disclosure, skilled artisans will appreciate that certain features are, for clarity, described herein in the context of separate embodiments, can also be provided in combination in a single embodiment. Conversely, various features that are, for brevity, described in the context of a single embodiment, can also be provided separately or in any sub-combination. Further, references to values stated in ranges include each and every value within that range.

The above discussion is meant to be illustrative of the principles and various embodiments of the present invention. Numerous variations and modifications will become apparent to those skilled in the art once the above disclosure is fully appreciated.

1. A face massaging device comprising a mask with at least one gua sha tool and at least one roller, wherein each of the at least one gua sha tool and the at least one roller automatically moves along a user's face.

2. The face massaging device of claim 1, wherein the automated movement is controlled by a controller.

3. The face massaging device of claim 2, wherein each of the at least one gua sha tool moves along a respective gua sha track on an inferior surface of the mask and each of the at least one roller moves along a respective roller track.

4. The face massaging device of claim 3, wherein each of the at least one gua sha tool is removably coupled to the respective gua sha track and each of the at least one roller is removably coupled to the respective roller track.

5. A face massaging device comprising:
a mask with a flexible shell conforming to a plurality of facial geometries;
at least one actuator disposed on the shell configured to deliver tactile stimulation;
internal motion-guiding channels oriented to direct the tactile stimulation from the at least one actuator from a wearer's mid-face to pre-auricular and cervical regions; and
an LED array positioned adjacent to at least one of the channels,
wherein a controller controls the at least one actuator to deliver the tactile stimulation that is then directed by the internal motion-guiding channels to generate wave-like stimulation; and
wherein the controller controls the LED array to provide light therapy that complements the wave-like stimulation.

6. The face massaging device of claim 5, wherein the at least one actuator comprises vibration motors located in the wearer's forehead, temple, cheek, nasolabial, jawline, and chin zones.

7. The face massaging device of claim 5, wherein the at least one actuator comprises inflatable micro-bladders fluidly coupled to a pump and valve array to generate sequential compression.

8. The face massaging device of claim 5, wherein the controller provides a plurality of preset massage and light therapy routines for the at least one actuator and the LED array.

9. The face massaging device of claim 5, wherein the LED array emits red and near-infrared wavelengths through a flexible diffuser.

10. The face massaging device of claim 5, wherein the mask has an inner layer adhered or otherwise affixed to the shell.

11. The face massaging device of claim 10, wherein the inner layer comprises a continuous silicone film that isolates the user from hard components.

12. The face massaging device of claim 10, wherein the inner layer has a skin-contacting surface and wherein the internal motion-guiding channels are located on the skin-contacting surface.

13. The face massaging device of claim 12, wherein the at least one actuator and the LED array are positioned between the outer shell and the inner layer.

14. The face massaging device of claim 5, further comprising a rechargeable lithium-ion battery and a USB-C charging interface coupled to a charge-management integrated circuit.

15. The face massaging device of claim 12, wherein the USB-C interface accepts five-volt input from standardized sources and includes over-voltage, over-current, and thermal protection.

16. The face massaging device of claim 15, wherein the USB-C interface has a receptacle that is recessed and sealed by an elastomeric gasket that maintains splash resistance.

17. The face massaging device of claim 5, wherein firmware limits heat and high-intensity modes while external power is connected.

18. The face massaging device of claim 5, wherein the at least one actuator comprises oscillating pads.

19. A method of cosmetic treatment comprising placing the face massaging device of claim 5 on a user, initiating a program that sequences stimulation from cheek to pre-auricular to cervical regions, and concurrently emitting light from the LED array.
